(19) United States
(12) Patent Application Publication
(10) Pub. No.: US 2026/0049052 A1
(43) Pub. Date: Feb. 19, 2026

(54) Methods of Synthesizing Chromophore Acceptors

(71) Applicant: Lightwave Logic, Inc., Englewood, CO (US)

(72) Inventors: Ginelle Ramann, Centennial, CO (US); Barry L. Johnson, Castle Rock, CO (US); Harvey F. Fulo, Denver, CO (US); Will D. Mikkelsen-Yi, Englewood, CO (US)

(73) Assignee: Lightwave Logic, Inc., Englewood, CO (US)

(21) Appl. No.: 19/299,874

(22) Filed: Aug. 14, 2025

Related U.S. Application Data
(60) Provisional application No. 63/683,982, filed on Aug. 16, 2024.

(51) Int. Cl. C07C 253/30

(52) U.S. Cl. CPC C07C 253/30

(57) ABSTRACT

The present disclosure is directed, in general, to synthesizing optionally substituted 2-dicyanomethylene-3-cyano-4-methyl-5-phenyl-5-perfluoromethyl-2,5-dihydrofuran comprising (i) reacting tributyl (1-ethoxyvinyl) tin with n-butyllithium at a temperature of between −30° C. and 10° C. to produce a first reaction product; (ii) reacting the first reaction product with 2,2,2-trifuoroacetophenone to produce a second reaction product: (iii) quenching the second reaction product with an acid to produce 3-hydroxy-3-phenyl-4,4,4-trifluoro-2-butanone; and (iv) reacting the 3-hydroxy-3-phenyl-4,4,4-trifluoro-2-butanone with malononitrile in the presence of base to produce 2-dicyanomethylene-3-cyano-4-methyl-5-phenyl-5-perfluoromethyl-2,5-dihydrofuran.

### CROSS REFERENCE TO RELATED APPLICATIONS

This application claims the benefit of U.S. Provisional Application Patent Application No. 63/683,982, filed Aug. 16, 2024. U.S. Provisional Application No. 63/683,982 is incorporated herein by reference in its entirety.

### BACKGROUND

The rise of silicon photonics has led to renewed interest in the use of electro-optic (EO) materials in next generation device applications. Materials with a strong EO response and high-speed phase modulation in thin film form are essential for low power and small footprint devices, including devices used in data acquisition systems, analog input/output modules (I/O modules), field transmitters, lab and field instrumentation, servo drive control modules, direct current (DC) power supply, alternating current (AC), and/or electronic load.

EO materials generally fall into three categories: (1) liquid crystals, including ferroelectric liquid crystals, and/or organic liquid crystals having a linear structure with a central core that contains several collinear rings, a linear unsaturated linkage and two terminal chains, and the like; (2) inorganic crystals characterized by a lack of inversion symmetry, such as KH₂PO₄ (KDP), KD₂PO₄ (KD*P or DKDP), lithium niobate (LiNbO₃), beta-barium borate (BBO), barium titanate (BTO), and (3) EO polymers, including non-linear optic (NLO) chromophore-polymer composite materials.

EO materials containing liquid crystals generally have desirable EO coefficient but exhibit inherently low phase modulation speeds due to the parasitic effect of the crystal metastructure. Conversely, EO materials containing lithium niobate and/or other inorganic crystals generally achieve desirable modulation speeds but their EO effects are inherently limited by optically active point defects invariably formed in the crystals during growth.

NLO chromophore-polymer composite materials can provide both high EO coefficient and high modulation speeds. Some non-linear optic (NLO) chromophore-polymer composite materials are of the general formula (I):

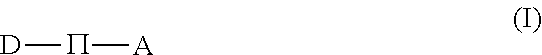

wherein D represents an organic electron-donating group; A represents an organic electron-accepting group having an electron affinity greater than the electron affinity of D; and II represents a II-bridge between the organic electron-accepting group and the organic electron-donating group. These NLO chromophore-polymer composite materials can provide both high EO coefficient and high modulation speeds. Methods for making some chromophores is hampered by low product yield. The methods for making some chromophore may include vinyl-lithiation and hydrolysis, as well as Knoevenagel condensations and Pinner reaction. See, for example, the synthetic method of the art depicted as follows as reported in He, M.; Leslie, T. M.; Sinicropi, J. A. Chem. Mater. 2002, 14, 2393-2400:

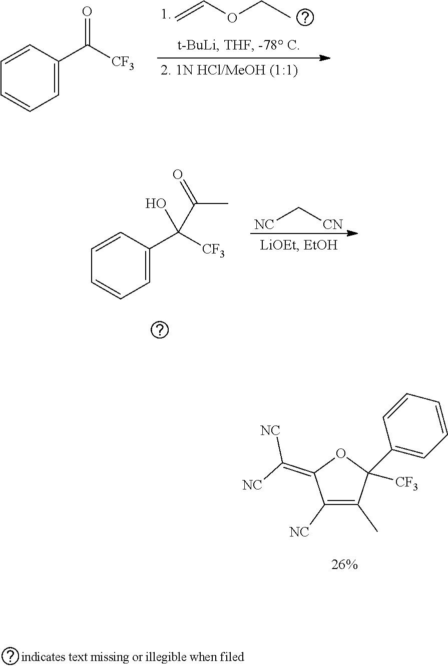

There is a need in the art for scalable, high-yield synthetic methods for producing the NLO chromophores and/or their constituents.

### BRIEF SUMMARY

The present disclosure is directed, in general, to methods of synthesizing optionally substituted 2-dicyanomethylene-3-cyano-4-methyl-5-phenyl-5-perfluoromethyl-2,5-dihydrofuran. In one aspect, the method comprises (i) reacting tributyl(1-ethoxyvinyl) tin with n-butyllithium at a temperature of between about −10° C. and about 10° C. to produce a first reaction product; (ii) reacting the first reaction product with 2,2,2-trifuoroacetophenone to produce a second reaction product; (iii) quenching the second reaction product with an acid to produce 3-hydroxy-3-phenyl-4,4,4-trifluoro-2-butanone; and (iv) reacting the 3-hydroxy-3-phenyl-4,4,4-trifluoro-2-butanone with malononitrile in the presence of base to produce 2-dicyanomethylene-3-cyano-4-methyl-5-phenyl-5-perfluoromethyl-2,5-dihydrofuran.

In some embodiments, the acid may be hydrochloric acid (HCl). In certain embodiments, the HCl may be at a concentration of about 5N to about 7N, or about 5N to about 6N, or about 6N to about 7N, or about 6N.

In certain embodiments, the temperature of step (i) may be between about −10° C. and about 10° C., or −5° C. and about 5° C., or about 0° C.

In some embodiments, step (i) may be performed in the presence of a solvent. In one embodiment, the solvent may be tetrahydrofuran (THF).

In certain embodiments, the base in step (iv) may be lithium carbonate.

In some embodiments, step (iv) may be performed in the presence of ethylene glycol.

Step (iv) may be performed at about 95° C. to about 105° C., or about 95° C. to about 100° C., or about 100° C. to about 105° C., or about 100° C.

In some embodiments, step (iv) may be performed with at least about 2.5 or 3 or 3.3 or 3.5 equivalents of malononitrile per equivalent of 3-hydroxy-3-phenyl-4,4,4-trifluoro-2-butanone.

An optional additional step in the process may be where the 3-hydroxy-3-phenyl-4,4,4-trifluoro-2-butanone produced in step (iii) contacted with dichloromethane, washed with brine and dried with MgSO₄ before step (iv).

In some embodiments, (i) the 2-dicyanomethylene-3-cyano-4-methyl-5-phenyl-5-perfluoromethyl-2,5-dihydrofuran compound may be independently substituted at one or more positions on the phenyl and/or methyl group with C1-C12 alkyl, aryl, heteroaryl, halogen, CF3, CN, or N, O, or S groups substituted with C1-C12 alkyl, aryl, heteroaryl groups, (ii) the phenyl group or the 4,4,4-trifluoromethyl group of 3-hydroxy-3-phenyl-4,4,4-trifluoromethyl-2-butone is substituted analogous to the phenyl group of 2-dicyanomethylene-3-cyano-4-methyl-5-phenyl-5-perfluoromethyl-2,5-dihydrofuran, and (iii) the terminal carbon of the carbon-carbon double bond of tributyl(1-ethoxyvinyl) tin is substituted analogous to the methyl group of the 2-dicyanomethylene-3-cyano-4-methyl-5-phenyl-5-perfluoromethyl-2,5-dihydrofuran compound.

Another aspect of the invention may be a nonlinear optical chromophore of the formula:

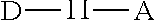

wherein D represents an organic electron-donating group; A represents an organic electron-accepting group having an electron affinity greater than the electron affinity of D; and II represents a II-bridge between A and D, wherein a compound disclosed herein acts as the acceptor group.

In some embodiments, the acceptor A of the nonlinear optical chromophore may be a substituted or unsubstituted 2-dicyanomethylene-3-cyano-4-methyl-5-phenyl-5-perfluoromethyl-2,5-dihydrofuran have the following structure:

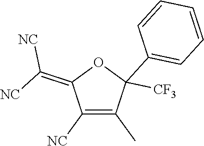 [SMILES: CC1=C(C#N)C(=C(C#N)C#N)OC1(c1ccccc1)C(F)(F)F]

It should be noted that it is contemplated that each of the elements above may be combined with any other elements.

### DETAILED DESCRIPTION

In some aspects, the disclosure concerns improved methods of synthesis of 2-dicyanomethylene-3-cyano-4-methyl-5-phenyl-5-perfluoromethyl-2,5-dihydrofuran compounds.

In one aspect, the method comprises (i) reacting tributyl(1-ethoxyvinyl) tin with n-butyllithium at a temperature of between about −30° C. and about 10° C. to produce a first reaction product; (ii) reacting the first reaction product with 2,2,2-trifuoroacetophenone to produce a second reaction product; (iii) quenching the second reaction product with an acid to produce 3-hydroxy-3-phenyl-4,4,4-trifluoro-2-butanone; and (iv) reacting the 3-hydroxy-3-phenyl-4,4,4-trifluoro-2-butanone with malononitrile in the presence of base to produce 2-dicyanomethylene-3-cyano-4-methyl-5-phenyl-5-perfluoromethyl-2,5-dihydrofuran.

In some embodiments, the acid is hydrochloric acid (HCl). In certain embodiments, the HCl is at a concentration of about 3N to about 8N or about 4N to about 7N or about 5N to about 7N or about 5.5N to about 6.5N or about 6N.

In certain embodiments, the temperature of step (i) is between about −20° C. and about 10° C., or −10° C. and about 5° C., or −5° C. and about 5° C., or about 0° C.

In some embodiments, step (i) is performed in the presence of a solvent. In one embodiment, the solvent is tetrahydrofuran (THF).

In certain embodiments, the base in step (iv) is lithium carbonate.

In some embodiments, step (iv) is performed in the presence of ethylene glycol.

Step (iv) may be performed at about 70° C. to about 130° C. or about 80° C. to about 120° C. or about 90° C. to about 110° C. or about 100° C.

In some embodiments, step (iv) is performed with at least about 2.5 or 3 or 3.3 or 3.5 equivalents of malononitrile per equivalent of 3-hydroxy-3-phenyl-4,4,4-trifluoro-2-butanone.

An optional additional step in the process may be where the 3-hydroxy-3-phenyl-4,4,4-trifluoro-2-butanone produced in step (iii) is contacted with dichloromethane, washed with brine and dried with MgSO₄ before step (iv). In certain embodiments, the reaction product of step (iii) may be eluted through a pad of silica gel. For example, the elution process may include first eluting the hexanes to remove tin byproduct and flushing with dichloromethane to elute the product.

In some embodiments, (i) the 2-dicyanomethylene-3-cyano-4-methyl-5-phenyl-5-perfluoromethyl-2,5-dihydrofuran compound may be independently substituted at one or more positions on the phenyl and/or methyl group with C1-C12 alkyl, aryl, heteroaryl, halogen, CF3, CN, or N, O, or S groups substituted with C1-C12 alkyl, aryl, or heteroaryl groups, (ii) the phenyl group or the 4,4,4-trifluoromethyl group of 3-hydroxy-3-phenyl-4,4,4-trifluoromethyl-2-butone may be substituted analogous to the phenyl group of 2-dicyanomethylene-3-cyano-4-methyl-5-phenyl-5-perfluoromethyl-2,5-dihydrofuran, and (iii) the terminal carbon of the carbon-carbon double bond of tributyl(1-ethoxyvinyl) tin may be substituted analogous to the methyl group of the 2-dicyanomethylene-3-cyano-4-methyl-5-phenyl-5-perfluoromethyl-2,5-dihydrofuran compound.

In certain embodiments, the phenyl or methyl groups of the 2-dicyanomethylene-3-cyano-4-methyl-5-phenyl-5-perfluoromethyl-2,5-dihydrofuran compound may be replaced with a different substituent such as optionally substituted C₁-C₁₂ alkyl, aryl and heteroaryl groups.

In certain embodiments, the 2-dicyanomethylene-3-cyano-4-methyl-5-phenyl-5-perfluoromethyl-2,5-dihydrofuran compounds may be useful in the construction of nonlinear optical chromophores. In particular, the compounds may function as an acceptor group in these constructs.

The nonlinear optical chromophores described herein may be applied in various electro-optic devices (e.g., nonlinear optical waveguide) in a variety of environments including those that function in environments where high photostability is important.

### Terms and Concepts

As used herein, the following terms have the following meanings unless expressly stated to the contrary.

As used herein, the term “about”, in the context of concentrations of components of the formulations or in property values, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

When values are expressed as approximations, by use of the antecedent “about,” it will be understood that the particular value forms another example.

All ranges are inclusive and combinable. In addition, when a range is recited, it is contemplated that all values within the range, including end points, are combinable in all possible combinations.

As used herein, the term “wt %” refers to weight percentage. The weight percentage of a component equals a ratio of a mass of a component to the total mass of the whole compound or product.

As used herein, the singular forms “a,” “an,” and “the” and similar referents in the context of describing the elements (especially in the context of the following claims) include plural references unless the context clearly dictates otherwise. For example, reference to “a substituent” encompasses a single substituent as well as two or more substituents, and the like. It is understood that any term in the singular may include its plural counterpart and vice versa, unless otherwise indicated herein or clearly contradicted by context.

Any use of section headings is intended to aid reading of the document and is not to be interpreted as limiting; information that is relevant to a section heading may occur within or outside of that particular section.

As used herein, the terms “for example,” “for instance,” “such as,” or “including” are meant to introduce examples that further clarify more general subject matter.

As used herein, the term “normal” (N) refers to the gram-equivalent weight of the solute per liter of solution. For example, 6N refers to a 6 gram-equivalent weight per liter of solution. 6N HCl is equivalent to 6M HCl.

As used herein, the term “molar” (M) refers to the number of moles of the solute per liter of solution.

As used herein, the term “electron-donating group” refers to an atom and/or a functional group that donates some of its electron density into a conjugated II system via resonance and/or inductive effects.

As used herein, the term “electron-accepting group” refers to an atom and/or a functional group that accepts some of the electron-donating group's electron density in a conjugated II system via resonance and/or inductive effects.

As used herein, the term “bridging group” refers to a functional group that bridges between the electron-donating group and the electron-accepting group in a conjugated II system.

As used herein, the term “compositions” refers to one or more mixed composition(s) that may include both a nonlinear electro-optic material and solvents.

As used herein, the term “electro-optic devices” refers to devices with electro-optical function that contain one or more resistive layer(s) described above. For example, the electro-optic devices may include electro-optic modulators (EOMs), which are optical devices in which a signal-controlled element exhibiting an electro-optic effect is used to modulate a beam of light.

As used herein, the term “nonlinear optical chromophore” (NLO Chromophore) refers to molecules or portions of a molecule that create a nonlinear electro-optic effect when irradiated with light. The chromophores are any molecular unit whose interaction with light gives rise to the nonlinear optical effect. The desired effect may occur at resonant or nonresonant wavelengths. The activity of a specific chromophore in a nonlinear electro-optic material is stated as its electro-optic coefficient (r33), which is related to the molecular dipole moment and hyperpolarizability. The various embodiments of NLO chromophores of the present disclosure are useful structures for the production of NLO effects.

Nonlinear optical chromophores in accordance with the various embodiments of the disclosure have the general formula (I):

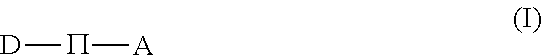

wherein D represents an organic electron-donating group; A represents an organic electron-accepting group having an electron affinity greater than the electron affinity of D; and ┌ represents a Π-bridge between A and D. The terms electron-donating group (donor or “D”), Π-bridge (bridging group or “Π”), and electron-accepting group (acceptor or “A”), and general synthetic methods for forming D-Π-A chromophores are well known in the art.

A donor is an atom or group of atoms that has a low oxidation potential, wherein the atom or group of atoms can donate electrons to an acceptor through a Π-bridge. The donor (D) has a lower electron affinity than the acceptor (A), so that, at least in the absence of an external electric field, the chromophore is generally polarized, with relatively less electron density on the donor (D). Typically, a donor group contains at least one heteroatom that has a lone pair of electrons capable of being in conjugation with the p-orbitals of an atom directly attached to the heteroatom such that a resonance structure can be drawn that moves the lone pair of electrons into a bond with the p-orbital of the atom directly attached to the heteroatom to formally increase the multiplicity of the bond between the heteroatom and the atom directly attached to the heteroatom (i.e., a single bond is formally converted to double bond, or a double bond is formally converted to a triple bond) so that the heteroatom gains formal positive charge. The p-orbitals of the atom directly attached to the heteroatom may be vacant or part of a multiple bond to another atom other than the heteroatom. The heteroatom may be a substituent of an atom that has π bonds or may be in a heterocyclic ring. Exemplary donor groups include but are not limited to R₂N—and RₙX¹—, where R is alkyl, aryl or heteroaryl, X¹ is O, S, P, Se, or Te, and n is 1 or 2. The donor group may be substituted further with alkyl, aryl, or heteroaryl.

An acceptor is an atom or group of atoms that has a low reductive potential, wherein the atom or group of atoms can accept electrons from a donor through a Π-bridge. The acceptor (A) has a higher electron affinity than the donor (D), so that, at least in the absence of an external electric field, the chromophore is generally polarized in the ground state, with relatively more electron density on the acceptor (D). Typically, an acceptor group contains at least one electronegative heteroatom that is part of a π bond (a double or triple bond) such that a resonance structure can be drawn that moves the electron pair of the π bond to the heteroatom and concomitantly decreases the multiplicity of the x bond (i.e., a double bond is formally converted to single bond or a triple bond is formally converted to a double bond) so that the heteroatom gains formal negative charge. The heteroatom may be part of a heterocyclic ring. Exemplary acceptor groups include but are not limited to —NO₂, —CN, —CHO, COR, CO₂R, —PO(OR)₃, —SOR, —SO₂R, and —SO₃R where R is alkyl, aryl, or heteroaryl. The acceptor group may be substituted further with alkyl, aryl, and/or heteroaryl.

A “Π-bridge” includes an atom or group of atoms through which electrons may be delocalized from an electron donor (defined above) to an electron acceptor (defined above) through the orbitals of atoms in the bridge. Such groups are very well known in the art. Typically, the orbitals will be p-orbitals on double (sp²) or triple (sp) bonded carbon atoms such as those found in alkenes, alkynes, neutral or charged aromatic rings, and neutral or charged heteroaromatic ring systems. Additionally, the orbitals may be p-orbitals on atoms such as boron or nitrogen. Additionally, the orbitals may be p, d or f organometallic orbitals or hybrid organometallic orbitals. The atoms of the bridge that contain the orbitals through which the electrons are delocalized are referred to here as the “critical atoms.” The number of critical atoms in a bridge may be a number from 1 to about 30. The critical atoms may be substituted with an organic or inorganic group. The substituent may be selected with a view to improving the solubility of the chromophore in a polymer matrix, to enhance the stability of the chromophore, or for other purposes.

### Examples

The instant disclosure is illustrated by the following non-limiting examples.

As used herein, the following names are associated with the shown structure: 2-dicyanomethylene-3-cyano-4-methyl-5-phenyl-5-perfluoromethyl-2,5-dihydrofuran:

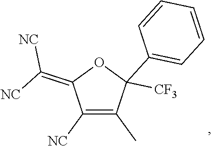 [SMILES: CC1=C(C#N)C(=C(C#N)C#N)OC1(c1ccccc1)C(F)(F)F]

3-hydroxy-3-phenyl-4,4,4-trifluoro-2-butanone:

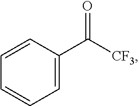 [SMILES: O=C(c1ccccc1)C(F)(F)F]

3-hydroxy-3-phenyl-4,4,4-trifluoro-2-butone:

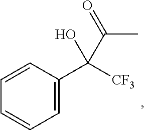

and tributyl(1-ethoxyvinyl) tin:

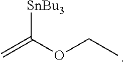

Synthesis of 2-dicyanomethylene-3-cyano-4-methyl-5-phenyl-5-perfluoromethyl-2,5-dihydrofuran

Tributyl(1-ethoxyvinyl) tin was contacted with n-butyllithium at a temperature of about 0° C. to produce a first reaction product. The first reaction product was then reacted with 2,2,2-Trifuoroacetophenone to produce a second reaction product. The second reaction product was then quenched with about 6N HCl to produce 3-hydroxy-3-phenyl-4,4,4-trifluoro-2-butanone. This reaction sequence is illustrated as follows:

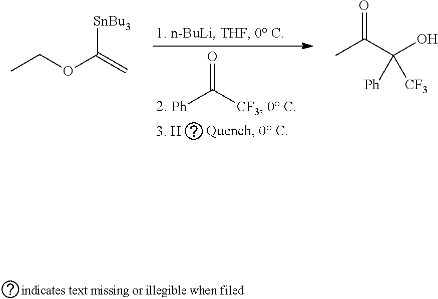

The 3-hydroxy-3-phenyl-4,4,4-trifluoro-2-butanone was reacted with malononitrile in the presence of lithium carbonate (Li₂CO₃) at a temperature of about 100° C. in ethylene glycol solvent to produce 2-dicyanomethylene-3-cyano-4-methyl-5-phenyl-5-perfluoromethyl-2,5-dihydrofuran. This reaction sequence is illustrated as follows:

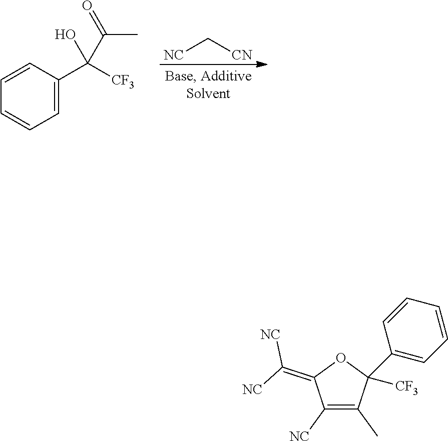

Yields for the production of 3-hydroxy-3-phenyl-4,4,4-trifluoro-2-butone using two instant processes are compared with those in the published art (He, et al. Chem. Matter. 2002, 14, 2393-2400) in Table 1.

TABLE 1
Production of 3-hydroxy-3-phenyl-4,4,4-trifluoro-2-butone

| | | Yield |
|---|---|---|
| Entry | Condition | (%) |
| 1 (art) | 2,2,2-trifuoroacetophenone (24.4 g), vinylether (2 eq), | 95.7 |
| | t-BuLi (2 eq), −78° C., quench = 1N HCl (aq) | |
| 2 | 2,2,2-trifuoroacetophenone (10 g), R-Sn (1.1 eq), n-BuLi | 78.3 |
| | (1.1 eq), 0° C., quench = 6N HCl (aq) | |
| 3 | 2,2,2-trifuoroacetophenone (33 g), R-Sn (1.1 eq), n-BuLi | 78.0 |
| | (1.1 eq), 0° C., quench = 6N HCl (aq) | |

Yields from the cyclization step (reacting the 3-hydroxy-3-phenyl-4,4,4-trifluoro-2-butanone with malononitrile in the presence of base to produce 2-dicyanomethylene-3-cyano-4-methyl-5-phenyl-5-perfluoromethyl-2,5-dihydrofuran) using the instant process compared to the published art (He, et al. Chem. Matter. 2002, 14, 2393-2400) are shown in Table 2.

TABLE 2
Cyclization Process Yields

| | | Yield |
|---|---|---|
| Entry | Condition | (%) |
| 1 (art) | 3-hydroxy-3-phenyl-4,4,4-trifluoro-2-butone (0.1 g), | 26 |
| | Malononitrile (2 eq), Li₂OEt (5 mol %), THE, reflux | |
| 2 | 3-hydroxy-3-phenyl-4,4,4-trifluoro-2-butone (100 g), | 74 |
| | Malononitrile (3.3 eq), Li₂CO₃ | |
| | (10 mol %), (CH₂OH)₂, 100° C. | |

Additional results comparing the procedure reported by He (He, et al. Chem. Matter. 2002, 14, 2393-2400) with the instantly disclosed synthetic scheme are reported in Table 3. The first step in Table 3 is the production of 3-hydroxy-3-phenyl-4,4,4-trifluoro-2-butone. The second step in Table 3 is the cyclization process.

TABLE 3
Comparison of yield from the instantly
disclosed process and that of the art.

| Procedure | 1^{St} step yield | 2^{nd} step yield | overall yield |
|---|---|---|---|
| Reported by He | 95.7% (140 | 26% (46 | 24.9% |
| | mmol scale) | mm scale) | overall |
| Instant inventors | 63% (100 | 52% (25 | 32.7% |
| repeating the He | mmol scale) | mmol scale) | overall |
| procedure | | | |
| Instantly disclosed | 92% (575 | 74% (575 | 68% |
| procedure | mmol scale) | mmol scale) | overall |

In some embodiments, the produced 3-hydroxy-3-phenyl-4,4,4-trifluoro-2-butanone produced may be contacted with dichloromethane or other appropriate solvent, washed with brine, and dried with MgSO₄ before the cyclization step.

The novel, highly-scalable synthesis of the instant disclosure offers several advantages over the known art including: (i) obviating the use of excess t-BuLi at cryogenic temperature (ii) facile vinyl-lithiation using vinyl-stannane and n-BuLi at about 0° C., (iii) fast and clean hydrolysis, and (iv) use of non-traditional solvent, ethylene glycol, exhibited excellent conversion of α-hydroxy ketone to the final product.

The instant methods use a lower equivalent of vinyl compound (e.g., about 1.1 eq R—Sn), a lower equivalent of a less-harsh base (e.g., about 1.1 eq n-BuLi), a higher temperature (e.g., about 0° C.), and a more productive about 6N HCl in the production of 3-hydroxy-3-phenyl-4,4,4-trifluoro-2-butone than the prior art. The instant methods also use a less-harsh base (lithium carbonate) in the cyclization step. In addition, the instant solvent (ethylene glycol) mimics the solvation of water without forming the common byproducts (furan and lactone). It is believed that this contributes to the instant high yield. Given the high yields achieved, it is further believed that the instant methods are scalable for commercial application.

What is claimed:

1. A method of synthesizing an optionally substituted 2-dicyanomethylene-3-cyano-4-methyl-5-phenyl-5-perfluoromethyl-2,5-dihydrofuran comprising
(i) reacting tributyl(1-ethoxyvinyl) tin with n-butyllithium at a temperature of between about −10° C. and about 10° C. to produce a first reaction product;
(ii) reacting the first reaction product with 2,2,2-trifuoroacetophenone to produce a second reaction product:
(iii) quenching the second reaction product with an acid to produce 3-hydroxy-3-phenyl-4,4,4-trifluoro-2-butanone; and
(iv) reacting the 3-hydroxy-3-phenyl-4,4,4-trifluoro-2-butanone with malononitrile in the presence of base to produce 2-dicyanomethylene-3-cyano-4-methyl-5-phenyl-5-perfluoromethyl-2,5-dihydrofuran.

2. The method of claim 1, wherein the acid is hydrochloric acid (HCl).

3. The method of claim 2, wherein the HCl is at a concentration of about 5N to about 7N.

4. The method of claim 2, wherein the HCl is at a concentration of about 6N.

5. The method of claim 1, wherein the temperature of step (i) is between about −5° C. and about 5° C.

6. The method of claim 1, wherein the temperature of step (i) is about 0° C.

7. The method of claim 1, wherein step (i) is performed in the presence of tetrahydrofuran (THF) solvent.

8. The method of claim 1, wherein the base in step (iv) is lithium carbonate.

9. The method of claim 1, wherein step (iv) is performed in the presence of ethylene glycol.

10. The method of claim 1, wherein step (iv) is performed at about 95° C. to about 105° C.

11. The method of claim 9, wherein step (iv) is performed at about 100° C.

12. The method of claim 1, wherein step (iv) is performed with at least about 2.5 equivalents of malononitrile per equivalent of 3-hydroxy-3-phenyl-4,4,4-trifluoro-2-butanone.

13. The method of claim 1, wherein step (iv) is performed with at least about 3 equivalents of malononitrile per equivalent of 3-hydroxy-3-phenyl-4,4,4-trifluoro-2-butanone.

14. The method of claim 1, wherein the 3-hydroxy-3-phenyl-4,4,4-trifluoro-2-butanone produced in step (iii) contacted with dichloromethane, washed with brine and dried with MgSO₄ before step (iv).

15. The method of claim 1, wherein (i) the 2-dicyanomethylene-3-cyano-4-methyl-5-phenyl-5-perfluoromethyl-2,5-dihydrofuran compound is independently substituted at one or more positions on the phenyl and/or methyl group with C1-C12 alkyl, aryl, heteroaryl, halogen, CF3, CN, or N, O, or S groups substituted with C1-C12 alkyl, aryl, heteroaryl groups (ii) the phenyl group or the 4,4,4-trifluoromethyl group of 3-hydroxy-3-phenyl-4,4,4-trifluoromethyl-2-butone is substituted analogous to the phenyl group of 2-dicyanomethylene-3-cyano-4-methyl-5-phenyl-5-perfluoromethyl-2,5-dihydrofuran, and (iii) the terminal carbon of the carbon-carbon double bond of tributyl(1-ethoxyvinyl) tin is substituted analogous to the methyl group of the 2-dicyanomethylene-3-cyano-4-methyl-5-phenyl-5-perfluoromethyl-2,5-dihydrofuran compound.
